(19) United States
(12) Patent Application Publication
(10) Pub. No.: US 2007/0009965 A1
(43) Pub. Date: Jan. 11, 2007

(54) Novel method for screening for GPCR agonists and/or antagonists from herbs

(75) Inventors: Li-Wei Hsu, Taichung City (TW); Su-Chen Chang, Taichung City (TW); Chen-Hsiang Shen, Taipei County (TW); Bang-Chi In, Taichung County (TW)

Correspondence Address: Scully, Scott, Murphy & Presser, 400 Garden City Plaza, Garden City, NY, 11530-3319, US

(73) Assignee: Advanced Gene Technology, Corp., Taichung City (TW)

(21) Appl. No.: 11/025,086

(22) Filed: Dec. 29, 2004

(51) Int. Cl. G01N 33/567; A61K 36/18

(52) U.S. Cl. 435/7.2; 424/725

(57) ABSTRACT

The present invention provides a novel method of using an herbal chip for screening for candidate compounds as a GPCR agonist or antagonist from herbs by using a labeled anti-GPCR-GPCR as a probe for hybridization.

### TECHNICAL FIELD OF THE INVENTION

The present invention relates to the field of drug discovery. Specifically, the present invention discloses a novel method for screening for candidate compounds as a GPCR agonist or antagonist from herbs by using an herbal chip.

### BACKGROUND OF THE INVENTION

U.S. Pat. No. 6,645,719 B2, one of the applicant's patents, disclosed an herbal chip comprising a plastic slide and a coating on the plastic slide which binds fractions or components obtained from herbs to said plastic slide in independently allocated microarrays on the coating, wherein said coating has active epoxy groups. U.S. Pat. No. 6,645,719 B2 also disclosed a method of using the herbal chip for screening for active ingredients contained in herbs, comprising loading a labeled probe(s)-containing solution onto the herbal chip for conducting hybridization, and imaging and identifying the microarrayed samples that react with or bind to the probe(s).

In an embodiment of U.S. Pat. No. 6,645,719 B2, tumor necrosis factor-α receptor (TNF-αR) was directly labeled with a fluorescent dye Cy3, and the resultant Cy3-labeled TNF-αR was added onto the herbal chip for use as a probe for conducting hybridization for screening for active ingredients contained in herbs that had a potential in biological activity similar to TNF-α or anti-TNF-αR antibody.

When trying to use G protein-coupled receptors (GPCRs), such as those of melanocortin receptor family, e.g. MC1 R, MC2R, MC3R, MC4R and MC5R, as a probe for conducting hybridization on the herbal chip for screening for active ingredients from the microarrayed fractions of herb extracts that may have a function on enhancing or inhibiting the activity of GPCRs, the inventors of the present invention has found that it was improper to directly label GPCRs with a fluorescent dye Cy3 or Cy5 for conducting hybridization on the herbal chip. In contrast to TNF-αR that could be expressed by a bacterial transformant and purified without destroying its tertiary structure or losing its biological function, and even that could be commercially purchased, GPCRs must be expressed by a mammalian cell line and, when substantially purified, will lose its tertiary structure and/or biological function. For sustaining the tertiary structure of GPCRs and retaining their biological function, GPCRs contained in mammalian cell membrane could be partially purified only, i.e. a GPCRs-containing sample obtained from partial purification would contain other unidentified protein impurities. When the GPCRs-containing sample was directly treated with Cy3 or Cy5, not only GPCRs but also the other unidentified protein impurities would be labeled. If the resultant Cy3- or Cy5-labeled GPCRs-containing sample was used as a probe for conducting hybridization on the herbal chip, falsely positive result(s) may be obtained when one or more of the Cy3- or Cy5-labeled unidentified protein impurities bind to ingredients in the microarrayed fractions of herb extracts on the herbal chip, i.e. the ingredients screened out were not the desired candidate compounds as a GPCR agonist or antagonist.

There was therefore a demand to develop an approach to overcome the above technical labeling problem caused by using GPCRs as a probe for conducting hybridization on the herbal chip.

### SUMMARY OF THE INVENTION

The present invention discloses a novel method for indirectly labeling a transmembrane protein, which could not be completely purified from cell membrane without destroying its tertiary structure or losing its biological function, with a dye or radioactive material, whereby the labeled transmembrane protein can be used as a probe for conducting hybridization on an herbal chip. In an embodiment of the present invention, the transmembrane protein is G protein-coupled receptors (GPCRs), such as those belonging to the melanocortin receptor family, e.g. MC1 R, MC2R, MC3R, MC4R and MC5R (hereinafter referred to as MCXR).

The present invention further provides a novel method for screening for candidate compounds as a GPCR agonist or antagonist from herbs by using an herbal chip.

### BRIEF DESCRIPTION OF THE DRAWINGS

FIG. 1 showed an image result of the binding of α-melanocyte stimulating hormone (NDP-MSH) to a MC3R-Cy3-anti-MC3R, MC4R-Cy3-anti-MC4R or MC5R-Cy3-anti-MC5R conjugate.

FIG. 2 showed an image result of the binding of ingredients in the fractions of a methanol extract of Cunninghamia laceolata (Lamb.) Hook to a Cy3-labeled anti-MC3R-MC3R conjugate.

FIG. 3 showed an intensity of fluorescent signal that was emitted by fraction numbers 54 to 58 in the test slide shown in FIG. 2.

### DETAILED DESCRIPTION OF THE INVENTION

G protein-coupled receptors (GPCRs) are G-protein-coupled transmembrane proteins located on the surface of mammalian cells, which have 7 transmembrane α-helixes and 6 loops connecting said 7 α-helixes, wherein each loop is connected to one α-helix and the 6 loops have different length of amino acids.

GPCRs play an important role in signal transduction of mammalian cells. GPCRs accept and transmit various signals from outside environment of cells by binding their specific ligands, and then the cells, when receiving the signals, will make necessary changes in their metabolism, activity and energy consumption in response to the change of outside environment. Different GPCRs accept their specific ligands for transmitting different outside signals. Up to now, it has been expected that there were approximately 650 GPCRs in human cell membrane, wherein more than 190 GPCRs had been identified. It has been reported that approximately 70 to 80% of signal transduction of mammalian cells depends upon activation of GPCRs.

Representatives of GPCRs are melanocortin receptors, e.g. MC1R, MC2R, MC3R, MC4R and MC5R, each of which binds α-, β- or γ-melanocyte stimulating hormone (MSH) or adrenocorticotrophin and is an adenyl cyclase.

Normally, there is just a native ligand or ligand conjugate for binding to and activating a GPCR. It has been found, however, that synthetic ligands that mimicked said native ligand or ligand conjugate could bind to its correspondent GPCR, and then could affect or change normal metabolism or function of mammalian cells. In therapy of GPCRs-involved diseases or conditions in mammals, such as endocrinopathy, cardiovascular and immunological disorders, those synthetic ligands were candidate drugs for treating or preventing various disorders associated with activation of GPCRs.

When applying the applicant's herbal chip as disclosed in U.S. Pat. No. 6,645,719 B2 and using GPCRs as a probe for screening for active ingredients from herbs that are mimic ligands that bind to GPCRs, the inventors of the present invention have found that GPCRs could not be completely purified from mammalian cell membrane without destroying its tertiary structure or losing its biological function, i.e. GPCRs in mammalian cell membrane could be partially purified only. A GPCRs-containing sample obtained from partial purification would contain other unidentified protein impurities. If the GPCRs-containing sample was directly treated with a fluorescent dye such as Cy3 and Cy5 or with a radioactive material, not only GPCRs but also the other unidentified protein impurities would be labeled. When the resultant labeled GPCRs-containing sample was used as a probe for conducting hybridization on the herbal chip, pseudo-positive result(s) may be obtained when one or more of the labeled unidentified protein impurities bind to ingredients in the microarrayed fractions of herb extracts on the herbal chip. The ingredients screened out were not the desired mimic ligands that bind to GPCRs, i.e. the desired candidate compounds as a GPCR agonist or antagonist could not be successfully screened out.

For overcoming the technical problem caused by using GPCRs as a probe for conducting hybridization on the herbal chip, the inventors of the present invention have found that monoclonal antibodies against C-terminal fragment of GPCRs, such as anti-MC1R, anti-MC2R, anti-MC3R, anti-MC4R and anti-MC5R, had been commercially available (Genx Bioscience Inc.), and said monoclonal antibodies could be labeled with a fluorescent dye, such as Cy3 and Cy5, or a radioactive material. Accordingly, the inventors of the present invention proposed a solution to solve the above technical problem by labeling said monoclonal antibodies (referred to as anti-MCXR, including anti-MC1R, anti-MC2R, anti-MC3R, anti-MC4R and anti-MC5R) with a dye or radioactive material, not said MCXR per se.

The present invention discloses a novel method for indirectly labeling GPCRs with a dye or radioactive material, comprising labeling monoclonal antibodies against C-terminal fragment of GPCRs with a dye or radioactive material and forming a complex of labeled anti-GPCR-GPCR (such as Cy3- or Cy5-anti-MCXR-MCXR), whereby the labeled anti-GPCR-GPCR can be used as a probe for conducting hybridization on the herbal chip for screening for candidate ligands as a GPCR agonist or antagonist.

Owing to the fact that GPCRs were partially purified from mammalian cell membrane and C-terminal fragment of GPCRs was used to bind to their monoclonal antibodies, the present invention uses N-terminal fragment of GPCRs and internal fragment of GPCRs that is located near N-terminus of GPCRs for binding to and screening for candidate ligands as a GPCR agonist or antagonist.

For conducting hybridization on the herbal chip for screening for candidate ligands as a GPCR agonist or antagonist, there were two alternatives: (1) labeled anti-GPCR could be firstly added to a solution containing GPCRs and unidentified protein impurities for forming a labeled anti-GPCR-GPCR complex, and then the resultant solution containing the labeled anti-GPCR-GPCR complex and non-labeled unidentified protein impurities could be added to the herbal chip, and (2) a solution containing GPCRs and unidentified protein impurities was firstly added to the herbal chip, followed by adding the labeled anti-GPCR thereby forming said labeled anti-GPCR-GPCR complex. In practice, the inventors of the present invention have found that the above approach (1) did not produce a satisfactory hybridization result, since the labeled anti-GPCR-GPCR complex was a big protein conjugate wherein the N-terminal fragment of its GPCR moiety did not successfully bind to the desired active ingredients in herb fractions microarrayed on the herbal chip, and/or the anti-GPCR moiety of said labeled anti-GPCR-GPCR complex possibly bound to undesired ingredients in the herb fractions, so that the undesired, pseudo-positive result(s) may occur.

Accordingly, the present invention provides a method of using an herbal chip for screening for candidate compounds as a GPCR agonist or antagonist from herbs, comprising the steps of:
(a) preparing an herbal chip as disclosed in U.S. Pat. No. 6,645,719 B2, wherein fractions of an herb extract were spotted in independently allocated microarrays on a coated plastic slide and wherein the coating has active epoxy groups that could bind to ingredients of herbs by reacting with their free OH, SH or NH₂ groups;
(b) loading a solution containing GPCRs and unidentified protein impurities onto the herbal chip for conducting hybridization;
(c) adding labeled anti-GPCR to form a labeled anti-GPCR-GPCR complex; and,
(d) imaging the hybridization result and identifying the herb fractions that react with or bind to the labeled anti-GPCR-GPCR complex.

In an embodiment of the present invention, fractions of a methanol extract of Cunninghamia laceolata (Lamb.) Hook were spotted in microarrays on a coated plastic slide having active epoxy groups on its surface for preparing an herbal chip, and labeled anti-MCXR-MCXR was added to the herbal chip for use as a probe for conducting hybridization on the herbal chip.

The following examples are used to further illustrate the technical content of the present invention. However, it should be noted that the present invention is not limited to the following examples.

### EXAMPLES

### Example 1

A coated plastic slide (1) having active epoxy groups on its surface was prepared in accordance with the method disclosed in the Paragraph entitled “Pretreatment of the Plastic Slide and Preparation of the Coated Plastic Slide” under the Example section of U.S. Pat. No. 6,645,719 B2.

In accordance with the teaching of the Paragraph entitled “Loading Samples onto the Coated Plastic Slide in Microarray Format” under the Example section of U.S. Pat. No. 6,645,719 B2, at least 25 ng/ml of α-melanocyte stimulating hormone (NDP-MSH, NH₂-Ser-Thr-Ser-Nle-Glu-His-D-Phe-Arg-Trp-Gly-Lys-Pro-Val-OH), that can bind to melanocortin receptors (MCXR), i.e. representatives of GPCRs, were spotted onto the coated plastic slide (1) by Microarrayer BioGrid I (BioRobotic, Cambridge, UK), and then the resultant slide (2) was washed with a blocking buffer (0.1 M sodium borate and 0.1 M ethanolamine) for removing unbound proteins.

In a dark room, under room temperature, at least 25 μg/ml of MC3R, MC4R and MC5R were individually added to the slides (2), and the resultant slides (3) were allowed to stand for 1 hour for completing hybridization. The slides (3) were washed with 1× TBST buffer (50 mM Tris, pH 7.3, 0.15 M NaCl and 0.02% Tween 20) and Milli-Q water, and then dried in an incubator at 37° C.

Also in the dark room, under room temperature, at least 5 μg/ml of Cy3-anti-MC3R, Cy3-anti-MC4R and Cy3-anti-MC5R were respectively added to the slides (3), each containing said bound MC3R, MC4R or MC5R. The resultant slides (4) were allowed to stand for 1 hour for completing the specific MCXR-anti-MCXR binding, and were then washed with 1× TBST buffer (50 mM Tris, pH 7.3, 0.15 M NaCl and 0.02% Tween 20) and Milli-Q water, and dried in an incubator at 37° C.

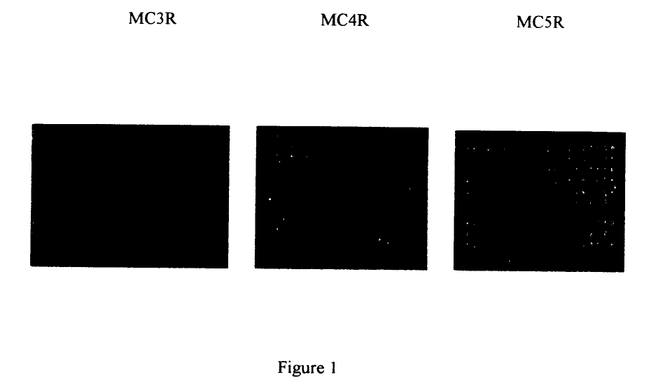

The dried slides (4) were imaged by a laser scanner (Axon, U.S.A.), and the image results were shown in FIG. 1. An analysis of fluorescence intensity of the fractions in said slides (4) was shown in the following Table 1.

TABLE 1

| | MC3R | MC4R | MC5R |
|---|---|---|---|
| Standard Deviation (SD) | 27.48 | 24.04 | 216.69 |
| Mean of Fluorescence | 97.00 | 58.50 | 722.50 |
| Intensity (M) | | | |
| Coefficient of Variance (CV)* | 28.33% | 41.09% | 29.99% |

*CV = SD/M × 100%

### Example 2

The experiment described above in Example 1 was repeated, except that (1) in replace of α-melanocyte stimulating hormone spotted onto the coated plastic slide (1), a methanol extract of Cunninghamia laceolata (Lamb.) Hook was fractionated by High Performance Liquid Chromatography (HPLC) into 96 fractions, which were then spotted onto the coated plastic slide (1) by the same Microarrayer BioGrid I, and the resultant slide was washed with the same blocking buffer for removing unbound proteins and compounds, and (2) only Cy3-labeled anti-MC3R-MC3R was used as a probe for conducting hybridization.

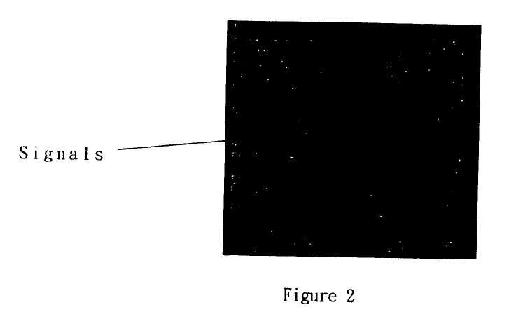

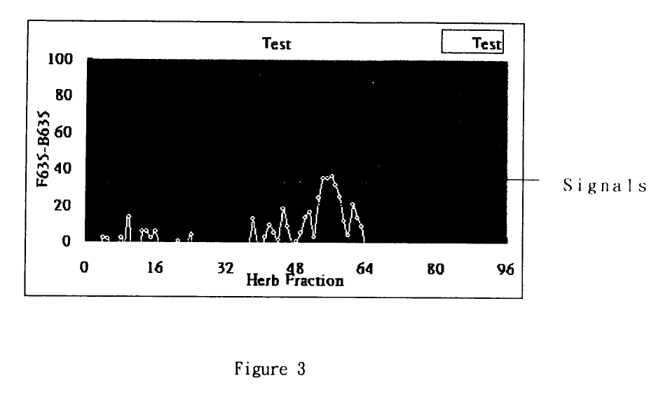

The resulting image result was shown in FIG. 2, and FIG. 3 showed that fraction numbers 54 to 58 in the test slide shown in FIG. 2 emitted a fluorescent signal, which indicated that there should be at least one candidate compounds in said herb fractions that bind to MC3R, which may have a biological activity as a MC3R agonist or antagonist.

What is claimed is:

1. A method for screening for candidate compounds as a G protein-coupled receptor (GPCR) agonist or antagonist from herbs by using an herbal chip, comprising the steps of:
(a) preparing a coated plastic slide having active epoxy groups on its surface;
(b) allocating fractions of herbs in microarrays on the coated plastic slide for preparing an herbal chip;
(c) loading a solution containing GPCRs and unidentified protein impurities onto the herbal chip for conducting hybridization;
(d) adding a labeled anti-GPCR to form a labeled anti-GPCR-GPCR complex on said herbal chip; and,
(e) imaging the hybridization result and identifying the herb fractions that react with or bind to the labeled anti-GPCR-GPCR complex.

2. The method of claim 1, wherein the GPCRs are melanocortin receptors.

3. The method of claim 2, wherein the melanocortin receptors are selected from the group of MC1R, MC2R, MC3R, MC4R and MC5R.

4. The method of claim 1, wherein the label is a dye or radioactive material.

5. The method of claim 1, wherein the anti-GPCR binds to C-terminal fragment of GPCR.

6. The method of claim 1, wherein N-terminal fragment of GPCR of said labeled anti-GPCR-GPCR complex binds to the candidate compounds in microarrayed fractions of herbs.
